(19) United States
(12) Patent Application Publication
(10) Pub. No.: US 2020/0075145 A1
(43) Pub. Date: Mar. 5, 2020

(54) MEDICAL EXAMINATION SUPPORT APPARATUS

(71) Applicant: FUJIFILM Corporation, Tokyo (JP)

(72) Inventors: Haruyasu NAKATSUGAWA, Tokyo (JP); Tsuyoshi HIRAKAWA, Tokyo (JP); Hiroshi HIRAMATSU, Tokyo (JP); Chuta KASAHARA, Tokyo (JP)

(73) Assignee: FUJIFILM Corporation, Tokyo (JP)

(21) Appl. No.: 16/554,156

(22) Filed: Aug. 28, 2019

(30) Foreign Application Priority Data
Aug. 31, 2018 (JP) 2018-163256

(51) Int. Cl. G16H 15/00; G16H 10/60; G16H 10/40

(52) U.S. Cl. CPC G16H 15/00; G16H 40/20; G16H 10/40; G16H 10/60

(57) ABSTRACT

A medical examination support apparatus includes: a display screen generation unit that generates a display screen for displaying identification information of a patient and a medical examination process so as to be associated with each other for each of a plurality of patients; and a medical examination process searching unit that compares a medical examination process corresponding to a selected patient to a medical examination process of a patient different from the selected patient in a case where there is an instruction from a request reception unit and searches for a medical examination process of a similar patient, which has a high similarity to the medical examination process corresponding to the selected patient. A searching result of the medical examination process of the similar patient is overlapped to a display screen such as a clinical flow screen.

### CROSS-REFERENCE TO RELATED APPLICATIONS

This application claims priority under 35 U.S.C § 119(a) to Japanese Patent Application No. 2018-163256 filed on Aug. 31, 2018. The above application is hereby expressly incorporated by reference, in its entirety, into the present application.

### BACKGROUND OF THE INVENTION

### 1. Field of the Invention

The present invention relates to a medical examination support apparatus.

### 2. Description of the Related Art

In the medical field, in order for medical staff members, such as doctors and laboratory technicians, to smoothly advance medical practices, examinations, and the like, an integrated medical examination support apparatus allowing the processes and results of medical examinations and the like to be shared by medical staff members, medical departments, or the like has been used. The medical examination support apparatus supports medical examinations, for example, by displaying a list in which the processes of medical examinations are listed for a plurality of patients so that the list is provided to medical staff members (JP2016-143205A, corresponding to US2016/224737A1). In addition, there is a medical examination support apparatus that supports medical examinations, for example, by displaying a so-called timeline, in which the processes of medical examinations of each patient are listed in time series for each patient, so that the medical examination processes of the patient are easily understood (JP2013-149267A).

On the other hand, in a case where a medical examination of a patient is performed or a medical examination process is checked, a doctor has to search for a medical examination process of a patient having similar conditions, such as a disease name, a symptom, and circumstances during hospitalization, and determine a new medical examination process based on the medical examination process of the similar patient. In order to help a searching operation of the doctor, a medical examination support apparatus has a function of searching for the similar patient and displaying information regarding the similar patient as a searching result (JP2017-049985A).

### SUMMARY OF THE INVENTION

However, in the medical examination support apparatus disclosed in JP2017-49985A, the doctor has to switch a display screen from a clinical flow screen to a screen searching for the similar patient, view the information of the similar patient based on the searching result, return to the clinical flow screen, and then think about the new medical examination process of a patient. As a consequence, it takes the doctor a lot of time and effort to input the medical examination process.

Therefore, it is an object of the invention to provide a medical examination support apparatus capable of saving time and effort in a case of searching for a medical examination process of a similar patient and easily performing an input of the medical examination process.

A medical examination support apparatus of the present invention comprises: a display screen generation unit; and a medical examination process searching unit, in which the display screen generation unit displays a searching result by the medical examination process searching unit on a display screen. The display screen generation unit generates the display screen for displaying identification information of a patient and a medical examination process so as to be associated with each other for each of a plurality of patients. The medical examination process searching unit compares a medical examination process corresponding to a selected patient selected from the plurality of patients to a medical examination process corresponding to a patient other than the selected patient and searches for a medical examination process corresponding to a similar patient, which has a high similarity to the medical examination process corresponding to the selected patient.

Displaying the searching result on the display screen means that the display screen is renewed for displaying the searching result on the display screen. In addition, mode of displaying the searching result includes a mode of displaying the searching result in an overlap manner with or side by side with the information indicating the identification information of the patient and the medical examination process so as to be associated with each other in the screen (window) displaying the information, and a mode of displaying the searching result in an overlap manner with or side by side with the information using another window other than the window displaying the above-mentioned information.

It is preferable that the medical examination support apparatus further comprises: a searching condition setting unit that sets a searching condition in a case of searching by the medical examination process searching unit and inputs the searching condition to the medical examination process searching unit, and the display screen generation unit displays the searching condition set by the searching condition setting unit on the display screen.

It is preferable that the display screen generation unit displays medical examination data configuring the medical examination process on the display screen, the searching condition setting unit sets the medical examination data as the searching condition, and the medical examination process searching unit searches for the medical examination process including medical examination data having a high similarity to the medical examination data set as the searching condition.

It is preferable that the medical examination process searching unit performs the searching by using similarity between pieces of medical examination data, the similarity being set by machine learning.

It is preferable that the medical examination data includes a process of future medical examination scheduled to be performed and a result of past medical examination performed.

It is preferable that the display screen generation unit generates a pictogram indicating the medical examination data and displays the pictogram as the medical examination data displayed on the display screen.

It is preferable that the display screen generation unit makes important medical examination data displayed in a different display mode from that of other medical examination data in the medical examination process.

It is preferable that a process of medical examination for the selected patient is predicted by using the searching result from the medical examination process searching unit.

It is preferable that a timeline for displaying a part or entirety of the medical examination process of the selected patient in a time series is created by using the searching result from the medical examination process searching unit.

It is preferable that a discharge destination of the selected patient is predicted by using the searching result from the medical examination process searching unit.

The medical examination support apparatus of the invention can save time and effort in a case of searching the medical examination process of the similar patient and easily perform the input of the medical examination process.

### BRIEF DESCRIPTION OF THE DRAWINGS

FIG. 1 is an explanatory diagram showing the configuration of a medical examination support system.

FIG. 2 is an explanatory diagram showing a client terminal provided in the medical examination support system.

FIG. 3 is a block diagram showing the configuration of the client terminal.

FIG. 4 is a block diagram showing the function of the client terminal.

FIG. 5 is a block diagram showing the configuration of a medical examination support apparatus.

FIG. 6 is a block diagram showing the function of the medical examination support apparatus.

FIG. 7 is a diagram showing an initial screen.

FIG. 8 is a diagram showing a clinical flow screen.

FIG. 9 is a partially enlarged view of the clinical flow screen.

FIG. 10 is a partially enlarged view of the clinical flow screen.

FIG. 11 is a diagram showing the clinical flow screen displayed a searching result in an overlapping manner.

FIG. 12 is a block diagram showing a function of a medical examination support apparatus of a second embodiment.

FIG. 13 is a diagram showing a clinical flow screen displaying a searching in an overlapping manner.

FIG. 14 is a diagram showing the clinical flow screen displaying a prediction result.

FIG. 15 is a diagram showing the clinical flow screen displayed a timeline in an overlapping manner.

### DESCRIPTION OF THE PREFERRED EMBODIMENTS

### First Embodiment

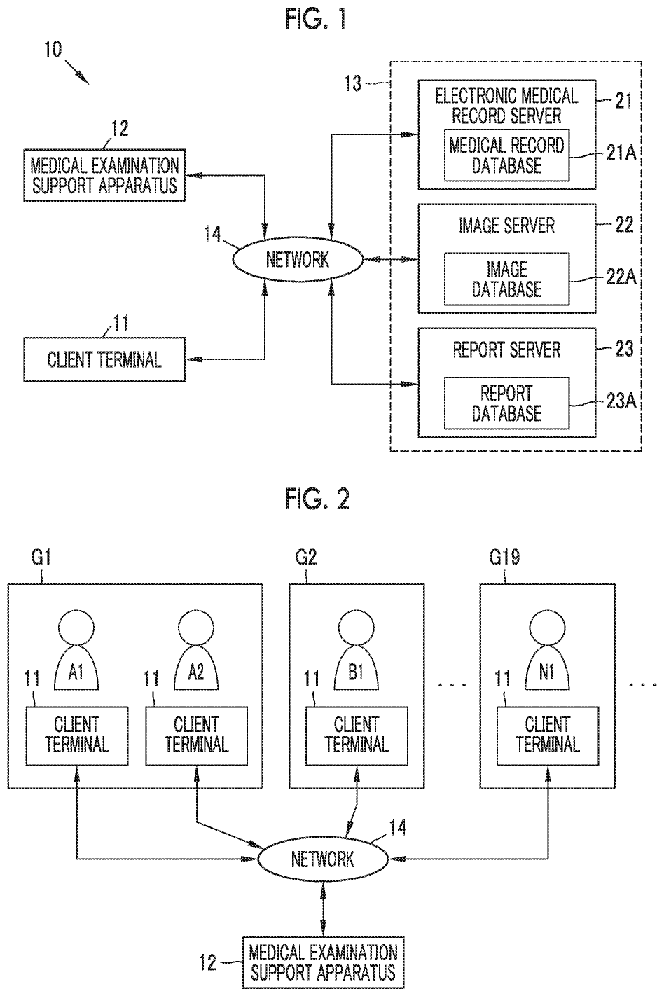

As shown in FIG. 1, a medical examination support system 10 is a computer system that performs medical examination support in a medical facility, such as a hospital, and comprises a client terminal 11, a medical examination support apparatus 12, and a server group 13. The respective elements configuring the medical examination support system 10 are communicably connected to each other using a network 14, such as a local area network (LAN) provided in the medical facility.

The client terminal 11 is a terminal for receiving a service provided from the medical examination support apparatus 12 (provided with a function of the medical examination support apparatus 12), and is a computer (including a tablet terminal or the like) directly operated by the medical staff member, such as a doctor, a laboratory technician, or a nurse. The client terminal 11 is installed in a medical department such as internal medicine or surgery, various examination departments such as a radiological examination department or a clinical examination department, a nurse center, or other necessary places. In addition, the client terminal 11 can be provided for each medical staff member, and can be shared by a plurality of medical staff members. Therefore, as shown in FIG. 2, the medical examination support system 10 includes a plurality of client terminals 11. For example, a group G1 is the “internal medicine” to which a doctor A1 and a doctor A2 belong, and the doctor A1 and the doctor A2 each have the client terminal 11. Similarly, for example, a group G2 is the “surgery” to which a doctor B1 belongs, and the group G2 includes at least one client terminal 11. In addition, for example, a group G19 is a “radiological department” to which a technician N1 belongs, and the group G19 includes at least one client terminal 11.

The medical examination support apparatus 12 provides a display screen, which includes medical examination data, to the client terminal 11 in response to a request from the client terminal 11, for example. The medical examination data is images, reports, and examination results acquired or created during medical practices, examinations, surgery, and the like, data obtained in the processes of other medical examinations or as results of the medical examinations, or information indicating the locations thereof (so-called link (alias) or the like). The medical examination support apparatus 12 acquires medical examination data to be used on the display screen from the server group 13.

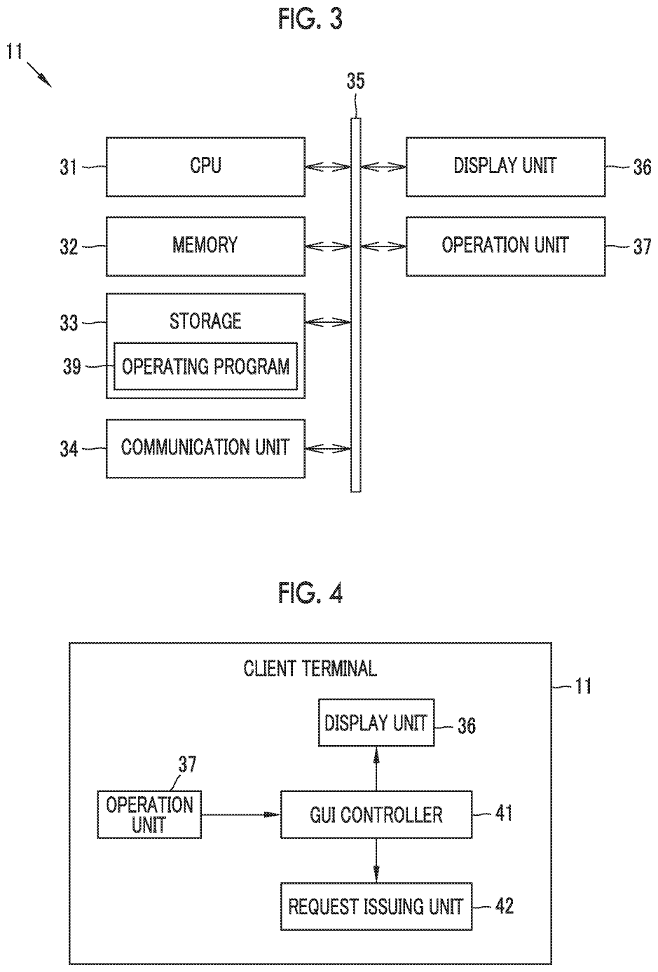

The display screen provided to the client terminal 11 by the medical examination support apparatus 12 refers to data used by the client terminal 11 to form a screen of a display unit 36 (refer to FIG. 3) of the client terminal 11. On the display screen provided to the client terminal 11 by the medical examination support apparatus 12, not only data for full screen display in which the client terminal 11 configures display of the entire screen but also data configuring display relevant to a part of the screen is included. For example, in the present embodiment, the medical examination support apparatus 12 provides the client terminal 11 with a display screen that can be displayed in a general window form in a part of the screen of the display unit 36.

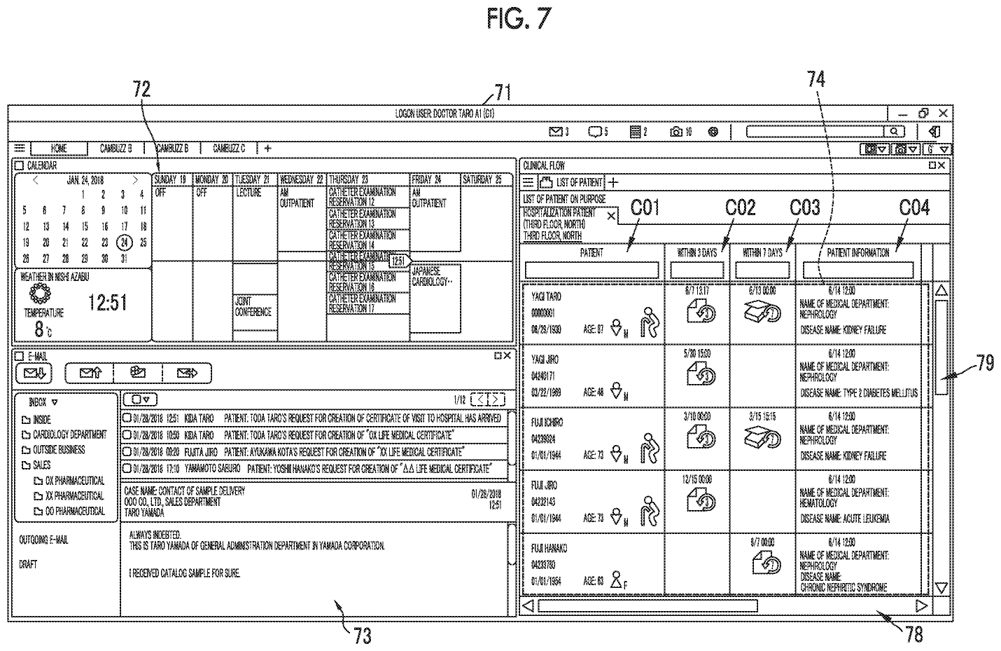

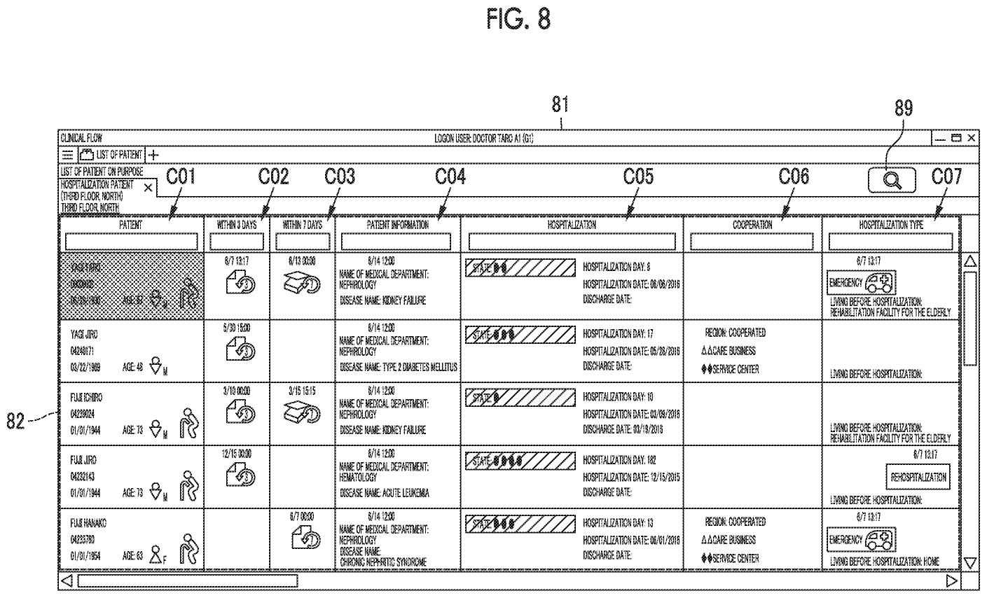

Specifically, the display screen provided to the client terminal 11 by the medical examination support apparatus 12 is an initial screen 71 (refer to FIG. 7), a clinical flow screen 81 (refer to FIG. 8), and the like. The clinical flow screen 81 is a display screen on which identification information of a patient and a part or entirety of a medical examination process are displayed so as to be associated with each other for each of a plurality of patients. The identification information of a patient is, for example, the patient's name, date of birth, age, and sex or identification data (ID), such as a unique number and/or symbol assigned to the patient, (hereinafter, referred to as a patient ID). The medical examination process refers to the process or result of past medical examination performed and future medical examination scheduled to be performed. Therefore, the medical examination process may include not only the medical examination data already acquired but also the medical examination data scheduled to be acquired. The medical examination data scheduled to be acquired is, for example, information regarding the presence or absence of an order of a specific examination, the scheduled date and time, and the type of medical examination data scheduled to be acquired.

The medical examination support apparatus 12 provides a display screen to the client terminal 11 in a description format using a markup language, such as an extensible markup language (XML) data, for example. The client terminal 11 displays a display screen in the XML format using a web browser. The medical examination support apparatus 12 can provide the client terminal 11 with a display screen in another format, such as JavaScript (registered trademark) object notation (JSON), instead of the XML.

The server group 13 searches for medical examination data in response to a request from the medical examination support apparatus 12, and provides the medical examination support apparatus 12 with the medical examination data corresponding to the request. The server group 13 includes an electronic medical record server 21, an image server 22, a report server 23, and the like.

The electronic medical record server 21 has a medical record database 21A that stores electronic medical records. The electronic medical record is a collection of one or a plurality of pieces of medical examination data. Specifically, the electronic medical records include medical examination data, such as medical practice records, results of sample examinations, a patient's vital signs, orders of an examination and the like, treatment records, and accounting data, for example. The electronic medical records can be input and viewed using the client terminal 11.

The medical practice record is a record of the content and the result of an inquiry or palpation or a disease name A sample is blood, tissue, or the like collected from a patient, and the sample examination is a blood test, a biochemical test, or the like. The vital signs are data indicating the state of a patient, such as the patient's pulse, blood pressure, or body temperature. The orders of an examination and the like are requests for an examination such as a sample examination, imaging using various modalities, creation of a report, treatment or surgery, medication, and the like. The treatment records are records of treatment, surgery, medication, and the like. The accounting data is data regarding a medical examination fee, a medicine fee, a hospitalization fee, and the like.

The image server 22 is a so-called picture archiving and communication system (PACS) server, and has an image database 22A that stores an examination image. The examination image is an image obtained by each of various image examinations, such as a computed tomography (CT) examination, a magnetic resonance imaging (MRI) examination, an X-ray examination, an ultrasound examination, and an endoscope examination. These examination images are recorded in a format based on the digital imaging and communications in medicine (DICOM) standard, for example. The examination image can be viewed using the client terminal 11.

The report server 23 has a report database 23A that stores an interpretation report. The interpretation report (hereinafter, simply referred to as a report) is a report that summarizes the interpretation results of examination images obtained by image examinations. The interpretation of an examination image is performed by a radiologist. A report can be created and/or viewed using the client terminal 11.

Each of the electronic medical record, the examination image, and the report described above includes a patient ID. In addition to the patient ID, the electronic medical record includes information for identifying the medical staff member, who has input medical examination data, for each piece of medical examination data. In addition to the patient ID, the examination image includes information for identifying the medical staff member (specifically, a laboratory technician) who has performed the examination. The report includes information for identifying the medical staff member (specifically, a radiologist) who has created the report. The information for identifying the medical staff member is the name of the medical staff member or an ID, such as a unique number and/or symbol assigned to each medical staff member (hereinafter, referred to as a medical staff ID).

Each of the client terminal 11, the medical examination support apparatus 12, and the servers 21 to 23 that configure the server group 13 is configured by installing an operating system program and an application program, such as a server program or a client program, on a computer as a base, such as a server computer, a personal computer, or a workstation. That is, the basic configurations of the client terminal 11, the medical examination support apparatus 12, and the servers 21 to 23 that configure the server group 13 are the same, and each of the client terminal 11, the medical examination support apparatus 12, and the servers 21 to 23 that configure the server group 13 comprises a central processing unit (CPU), a memory, a storage, a communication unit, and a connection circuit for connecting these to each other. The communication unit is a communication interface (LAN port or the like) for connection with the network 14. The connection circuit is, for example, a motherboard for providing a system bus and/or a data bus and the like.

As shown in FIG. 3, the client terminal 11 comprises a CPU 31, a memory 32, a storage 33, a communication unit 34, a connection circuit 35, the display unit 36, and an operation unit 37. The display unit 36 is a display using, for example, liquid crystal, and has a screen for displaying at least a display screen provided by the medical examination support apparatus 12. The operation unit 37 is, for example, a pointing device such as a mouse and/or an input device such as a keyboard. The display unit 36 and the operation unit 37 can configure a so-called touch panel.

In addition to the operating system program and the like, the client terminal 11 stores an operating program 39 in the storage 33. The operating program 39 is an application program for receiving the function of the medical examination support apparatus 12 using the client terminal 11. In the present embodiment, the operating program 39 is a program of a web browser. However, the operating program 39 can be a dedicated application program for receiving the provision of the function of the medical examination support apparatus 12. The operating program 39 may include one or a plurality of gadget engines for controlling a part or entirety of the display screen provided by the medical examination support apparatus 12. The gadget engine is a subprogram that realizes various functions by being operated in conjunction with a web browser or the like.

In a case where the operating program 39 is activated in the client terminal 11, as shown in FIG. 4, the CPU 31 of the client terminal 11 cooperates with the memory 32 to function as a graphical user interface (GUI) controller 41 and a request issuing unit 42.

The GUI controller 41 displays a display screen provided by the medical examination support apparatus 12 on the web browser in the display unit 36. The GUI controller 41 controls the client terminal 11 according to an operation instruction that is input using the operation unit 37, for example, by a button clicking operation using a pointing device.

The request issuing unit 42 issues a request (hereinafter, referred to as a processing request) for various processes with respect to the medical examination support apparatus 12 according to an operation instruction of the operation unit 37. Examples of the processing request issued by the request issuing unit 42 include a display screen distribution request, a display screen editing request, or a searching result displaying request. The request issuing unit 42 transmits a processing request to the medical examination support apparatus 12 through the communication unit 34 and the network 14.

The display screen distribution request requests the medical examination support apparatus 12 to distribute a display screen having a specific configuration. For example, any one of the initial screen 71, the clinical flow screen 81, and the like can be designated and distributed in response to the display screen distribution request.

The display screen editing request is for requesting the medical examination support apparatus 12 to edit the content of medical examination data and the like to be displayed on the display screen after receiving the distribution of the display screen having a specific configuration from the medical examination support apparatus 12. For example, in the case of receiving the distribution of the clinical flow screen 81, a request for designation or change of a list of patients to be displayed, a request for designation or change of a display target period of the medical examination process, a request for designation or change of the medical examination process to be displayed, or a request for sorting the display content is the display screen editing request.

The display screen distribution request and/or the display screen editing request, the searching result displaying request, and the like include information, such as a medical staff ID and the address of the client terminal 11 on the network. The medical staff ID is input on the login screen (not shown) to the medical examination support system 10 (or the medical examination support apparatus 12).

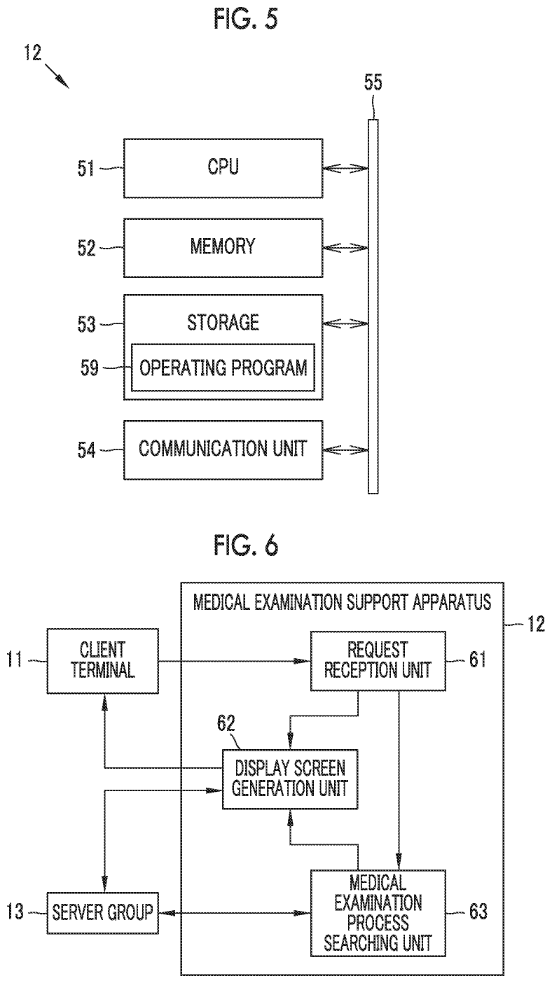

As shown in FIG. 5, the medical examination support apparatus 12 comprises a CPU 51, a memory 52, a storage 53, a communication unit 54, and a connection circuit 55. The medical examination support apparatus 12 can comprise a display unit and/or an operation unit as necessary, similarly to the client terminal 11. In the present embodiment, the medical examination support apparatus 12 does not have a display unit and an operation unit although the display unit and/or the operation unit can be attached as necessary.

In addition to the operating system program and the like, the medical examination support apparatus 12 stores an operating program 59 in the storage 53. The operating program 59 is an application program for making a computer configuring the medical examination support apparatus 12 function as the medical examination support apparatus 12. In a case where the operating program 59 is activated, as shown in FIG. 6, the CPU 51 of the medical examination support apparatus 12 cooperates with the memory 52 to function as a request reception unit 61, a display screen generation unit 62, a medical examination process searching unit 63, and the like.

The request reception unit 61 receives various processing requests, such as a display screen distribution request and a display screen editing request, from the client terminal 11. In a case where the request reception unit 61 receives various processing requests, the request reception unit 61 inputs a processing instruction to each unit, which performs the corresponding processing, according to the content of the requested processing. For example, in a case where there is a display screen distribution request from the client terminal 11, the request reception unit 61 inputs an instruction to generate the corresponding display screen to the display screen generation unit 62. Similarly, in a case where there is a display screen editing request from the client terminal 11, the request reception unit 61 inputs an instruction to edit the corresponding display screen to the display screen generation unit 62. The request reception unit 61 also receives a request for login to the medical examination support apparatus 12, and a login processing unit (not shown) performs login processing, such as checking of a medical staff ID and a password.

The display screen generation unit 62 generates or edits various display screens, such as the clinical flow screen 81. In the present embodiment, in a case where there is a new display screen distribution request, a display controller 66 generates XML data indicating the display screen, and in a case where there is a display screen editing request, the display controller 66 edits previously created XML data according to the request content. The display screen generation unit 62 accesses the server group 13 as necessary, and acquires information regarding the medical examination process and the like used for generation or editing of the display screen. In order to reduce the frequency of access to the server group 13, the display screen generation unit 62 can hold some or all of the pieces of information regarding the medical examination process and the like acquired from the server group 13. In a case where the login processing unit normally completes the login processing, the display screen generation unit 62 generates an initial screen 71 (refer to FIG. 7) to be displayed first after login. At the time of creating or editing the initial screen 71, the display screen generation unit 62 acquires information required for generation or editing of the initial screen 71 from the server group 13, the client terminal 11, or other apparatuses or systems that cooperate with the medical examination support system 10.

The medical examination process searching unit 63 searches for a medical examination process of a similar patient in a case where there is an instruction from a request reception unit 61. Specifically, the medical examination process searching unit 63 compares a medical examination process corresponding to a patient selected from a plurality of patients satisfying predetermined conditions to medical examination processes of patients different (other patients) from the selected patient and searches for a medical examination process corresponding to a patient (similar patient) having a high similarity to the selected patient in the medical examination processes.

The medical examination process searching unit 63 acquires information regarding the medical examination process corresponding to the selected patient (in the present embodiment, medical examination data configuring medical examination process), in a case of receiving an instruction of a searching process from the request reception unit 61. For the information (medical examination data), in a case where the medical examination process searching unit 63 uses the information regarding the medical examination process and the like, the display screen generation unit 62 may use information in which the medical examination support apparatus 12 retains or may acquire it directly from the server group 13 for use in generating, editing, and/or renewing the display screen.

Then, from the information acquired as described above (hereinafter, referred to as medical examination data), the medical examination process searching unit 63 performs the searching process for the similar patient including medical examination data having a high similarity to the medical examination data of the selected patient by using a medical examination data set as a searching condition. In the searching process, for example, comparison with the disease name of the selected patients is performed in a case where an item of the medical examination data set as the searching condition is a disease name. Then, for the item of the disease name, the medical examination process having the medical examination data with high similarity is searched for by comparing the medical examination data of the selected patient to past medical examination data.

The medical examination process searching unit 63 outputs the searching result for the medical examination process of the similar patient to the display screen generation unit 62. The display screen generation unit 62 renews the display screen by overlapping the searching result of the medical examination process searching unit 63 to a display screen of the clinical flow screen 81 and the like. That is, the medical examination process of the selected patient and the medical examination process of the similar patient which is narrowed by the searching process of the medical examination process searching unit 63 are displayed on a single display screen. In the following description, “renewing the display screen by overlapping” the information such as the examination result described above on the display screen may mean “displaying in an overlapping manner”, “displaying by overlapping”, or simply “overlapping”.

Although the present embodiment is described by an example in which a display mode of the information of the searching result and the like is an overlap-displaying mode, in the present invention, the medical examination process of the selected patient and the medical examination process of the similar patient may be displayed on a single display screen. For this reason, it may be a mode for displaying the medical examination process of the similar patient in an overlapping or parallel disposing manner to the medical examination process of the selected patient in a window which displays the medical examination process of the selected patient. In addition, it may be a mode for popping up a window for displaying the medical examination process of the similar patient and displaying the window in an overlapping or parallel-disposing manner to the window for displaying the medical examination process of the selected patient, independently of the window which displays the medical examination process of the selected patient.

The medical examination support system 10 configured as described above operates as follows. First, in a case where a medical staff member logs in to the medical examination support system 10 using the client terminal 11, the display screen generation unit 62 generates the initial screen 71 shown in FIG. 7 based on the setting or the like for each medical staff member and provides the initial screen 71 to the client terminal 11. Thus, the client terminal 11 displays the initial screen 71 on the screen of the display unit 36.

The initial screen 71 has, for example, three display fields of a schedule display field 72, an e-mail display field 73, and a list display field 74. The display content of the schedule display field 72 and the e-mail display field 73 is generated by acquiring information from an apparatus or a system other than the client terminal 11 by the gadget engine, which is a part of the operating program 39 of the client terminal 11. In the present embodiment, the list display field 74 displays at least a part of the clinical flow screen 81. Therefore, the display screen generation unit 62 generates the initial screen 71 including the schedule display field 72 and the e-mail display field 73, which do not include the content, and the list display field 74 which includes the content of the clinical flow screen 81. The client terminal 11 displays the initial screen 71, on which the content of the schedule display field 72 and the e-mail display field 73 is supplemented, on the screen of the display unit 36 using the gadget engine.

In a case where all of the content to be displayed does not fit in the list display field 74, a scroll bar 78 and a scroll bar 79 for transitioning (so-called scrolling) the display content of the list display field 74 are displayed in the list display field 74 or in the vicinity of the list display field 74. The scroll bar 78 is a GUI operated to transition the display content of the list display field 74 in a horizontal direction to display a non-displayed portion. The scroll bar 79 is a GUI operated to transition the display content of the list display field 74 in a vertical direction to display a non-displayed portion. Display and control of such a GUI are performed by the GUI controller 41.

For example, in a case where a predetermined menu or the like is operated using a GUI of a pointing device (not shown) and the like, on the initial screen 71 described above, the request issuing unit 42 issues a display screen distribution request. The operation in which the request issuing unit 42 issues a display screen distribution request based on the operation of such a GUI or the like is a display screen distribution request step. In the present embodiment, in order to display the entire clinical flow screen 81 partially displayed in the list display field 74, an operation for displaying the clinical flow screen 81 is performed. As a result, the request issuing unit 42 issues a distribution request of the clinical flow screen 81.

In a case where the request issuing unit 42 issues a display screen distribution request, the request reception unit 61 in the medical examination support apparatus 12 receives the display screen distribution request, and the display screen generation unit 62 generates a display screen according to the display screen distribution request. The operation of the display screen generation unit 62 for generating a display screen according to the display screen distribution request is a display screen generation step. In the present embodiment, the display screen generation unit 62 generates the clinical flow screen 81 using information according to the medical examination process or the like appropriately acquired from the server group 13 or the like.

As described above, in a case where the client terminal 11 issues the searching result displaying request and the request reception unit 61 receives the request, the medical examination process searching unit 63 performs the searching process. In a case where the client terminal 11 issues the searching result displaying request, first, one patient is selected from a plurality of patients displayed on the display screen such as the clinical flow screen 81.

As shown in FIG. 8, the clinical flow screen 81 generated in the present embodiment comprises a clinical flow display field 82 having a patient column C01, a three-day medical examination result column C02, a seven-day medical examination result column C03, an patient-information column C04, a hospitalization-state column C05, a cooperation information column C06, a hospitalization type column C07, and the like, for example. The patient column C01 configures a list of a plurality of patients satisfying predetermined conditions. As an example of the predetermined conditions, a condition that a person is an inpatient, that a person in charge is a medical staff member who is a login user, or that a person in charge is a group (medical department or the like) to which a medical staff member who is a login user belongs is included.

Each of the three-day medical examination result column C02, the seven-day medical examination result column C03, the patent-information column C04, the hospitalization-state column C05, the cooperation information column C06, and the hospitalization type column C07 is a collection of fields for displaying each process or result regarding a medical examination. Each column of the three-day medical examination result column C02, the seven-day medical examination result column C03, the patient-information column C04, the hospitalization-state column C05, the cooperation information column C06, the hospitalization type column C07, and the like (columns other than the patient column C01) is a medical examination process column for displaying the medical examination process. In the clinical flow display field 82, a non-displayed portion can be displayed by a scroll operation or the like, similarly to the list display field 74 on the initial screen 71.

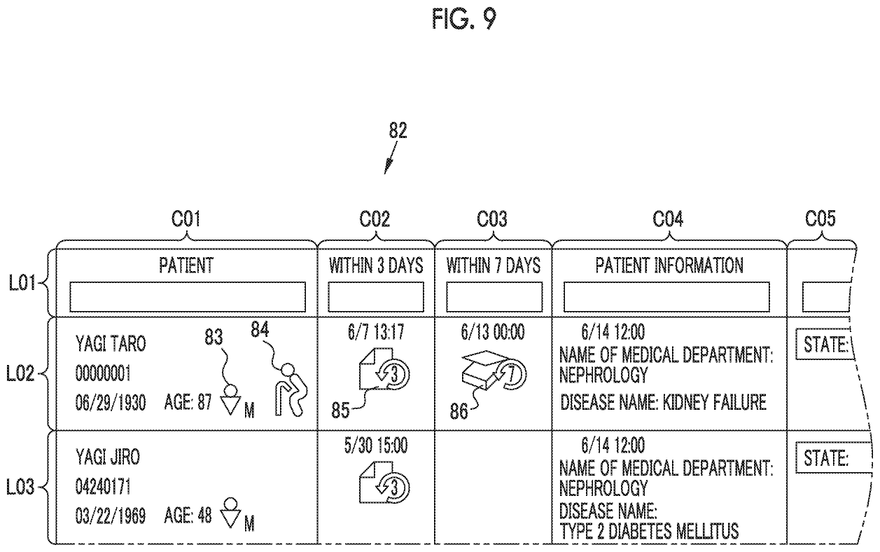

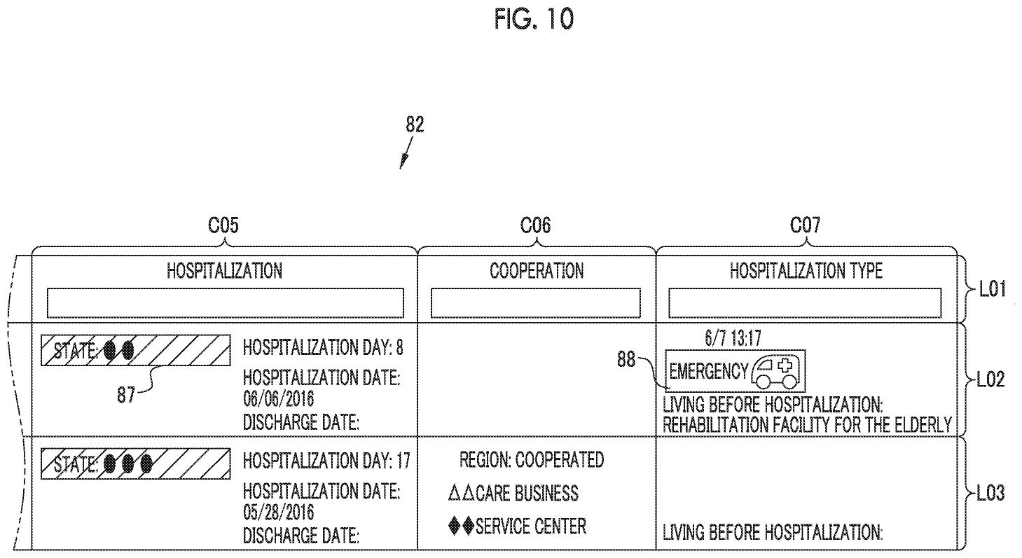

Second and subsequent rows in the clinical flow display field 82 configure a so-called clinical flow in which identification information of each patient is associated with the medical examination process of the patient. In FIGS. 9 and 10, clinical flows for two patients, that is, “Yagi Taro” shown in the row L02 and “Yagi Jiro” shown in the row L03 are exemplified.

The medical examination process included in the clinical flow of each patient is displayed for each item (for example, for each medical examination data) included in each medical examination process. As shown in FIG. 9, a pictogram 83 showing sex and a pictogram 84 showing that the patient is an aged person are attached to the patient column C01 in addition to the patient's name, identification information, the date of birth, age, and the like. In addition, a pictogram showing a medical examination result issued within 3 or 7 days is attached to the medical examination three-day result column C02 and the seven-day medical examination result column C03. In a case of “Yagi Taro”, a pictogram 85 showing that it is difficult to be discharged from the hospital within 3 days and a pictogram 86 showing that a family interview or a conference has been conducted within 7 days are attached. In addition, a name of a medical department or a disease name is input in the patient-information column C04.

As shown in FIG. 10, a pictogram 87 showing a health state of the patient is attached to the hospitalization-state column C05 in addition to a date and days of hospitalization. A pictogram 87, for example, shows a seriousness of the health state of the patient as the number of circles of a rectangular frame. In addition, since the pictogram 87 shows important medical examination data, it is displayed in a display mode different from the other medical examination data. In FIGS. 8 and 10, although a difference in the display mode is represented by presence or absence of a hatching for the convenience of drawing, the display mode is not limited thereto. The display mode may be a display mode which can distinguish between the important medical examination data and the other medical examination data, for example, using a different color in the pictogram 87. In addition, a facility name is input in the cooperation information column C06 in a case where a medical examination is performed in cooperation with other facilities. In addition, a pictogram showing states during hospitalization, information of a living before hospitalization, and the like are input in the hospitalization type column C07. In the case of “Yagi Taro”, a pictogram 88 showing an emergency hospitalization by ambulance transportation and the like is attached.

In the present embodiment, one of a plurality of patients displayed on the patient column C01 is selected as the selected patient. An example shown in FIG. 8 shows a state in which, among a plurality of patients, “Yagi Taro” is selected, for example, by a button clicking operation using a pointing device. In addition, in order to show the selected patient, only a display field of “Yagi Taro” in the patient column C01 is displayed in a different display mode. In FIG. 8, although a difference in the display mode is represented by presence or absence of a dot pattern for the convenience of drawing, the display mode is not limited thereto. The display mode may be a display mode which can distinguish between the selected patient and the other patients, for example, using different colors in the display fields, or changing a thickness of a frame surrounding the display field.

From the state in which the selected patient is selected as shown in FIG. 8, a user performs an input operation of the searching process. The input operation of the searching process is performed, for example, by a clicking operation of a searching button 89. In a case where the input operation of the searching process is performed, the client terminal 11 issues the searching result displaying request. The searching result displaying request includes information of a patient to be selected on the display screen.

In a case where the request reception unit 61 receives the searching result displaying request from the client terminal 11, the request reception unit 61 inputs the information of the selected patient and the instruction of searching for the medical examination process of the similar patient to the medical examination process searching unit 63. In the example shown in FIG. 8, information of “Yagi Taro” selected as the selected patient and the instruction of the searching process are input to the medical examination process searching unit 63. In this case, the medical examination process searching unit 63 acquires a medical examination data configuring a medical examination process corresponding to “Yagi Taro”. Then, the medical examination process searching unit 63 performs a searching processing of searching for a medical examination process including medical examination data which has a high similarity to the medical examination data set as the searching condition. In this case, a medical examination process of a patient who fulfills the predetermined conditions similar to the selected patient can also be a searching target.

In the example shown in FIG. 8, a preset item of the medical examination data as a searching condition is a disease name Then, by comparing the medical examination data of “Yagi Taro” which is the selected patient with medical examination data accumulated in the past, for an item of a disease name, a medical examination process having a high similarity to “kidney failure” is searched for.

The similarity between pieces of medical examination data, for example, is set by machine learning, and from the past cases, the similarity between the disease names having close medical examination processes are set to be high, and the similarity between the disease names having completely different medical examination processes are set to be low. The machine learning method of setting the similarity is preferable to use artificial intelligence (AI) and the like.

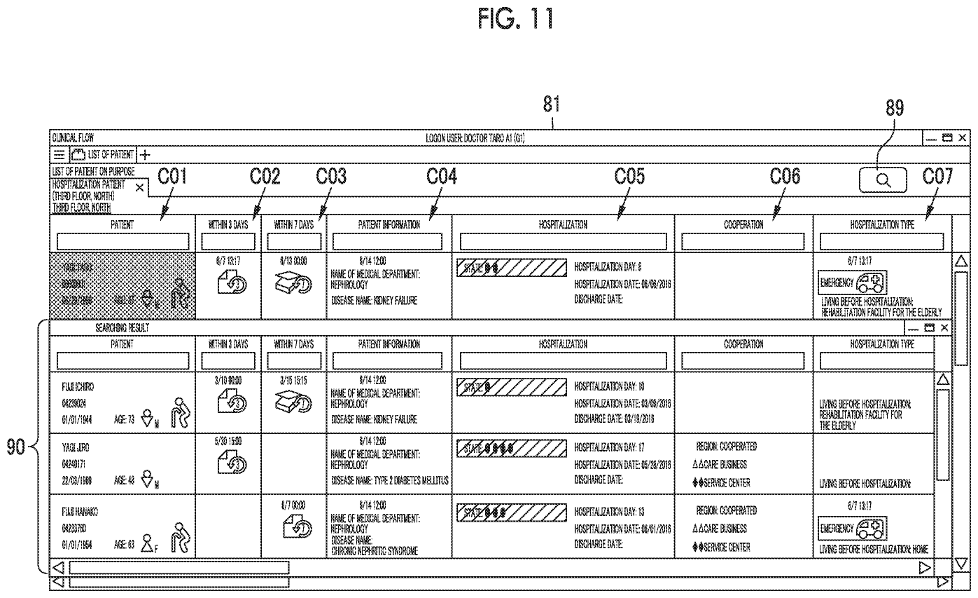

The medical examination process searching unit 63 inputs the searching result of the above-mentioned searching process to the display screen generation unit 62. Then, the display screen generation unit 62 overlaps the searching result of the medical examination process searching unit 63 on the display screen. In the case of performing the searching process in the example shown in FIG. 8, as shown in FIG. 11, the searching result 90 obtained by the searching is displayed in the overlapping manner on the clinical flow screen 81. In the searching result 90 medical examination processes of “Fuji Ichiro”, “Yagi Jiro”, and “Fuji Hanako” are displayed in descending order of similarity of disease name.

As described above, in a case where the searching result 90 is displayed in the overlapping manner on the clinical flow screen 81, the medical examination process of “Yagi Taro” which is the selected patient and the medical examination processes of “Fuji Ichiro”, “Yagi Jiro”, and “Fuji Hanako” which are the similar patients narrowed by the searching process are displayed on a single display screen. Therefore, the medical examination processes become easier to be compared with each other, and in a case where there is an item that is not entered or that is needed to be corrected, the medical examination process becomes easier to be created by using the medical examination data of the similar patients as reference. In addition, since a plurality of items configuring the medical examination process can be compared with each other on a single display screen, for example, even in a case where the disease name is the same, a consideration can be taken that a similar patient with a difference in hospitalization period is excluded from examination. However, in a case where the display screen and the search screen are separate from each other as in the method in the related art, after performing the searching process by using the disease name as the searching condition, the searching condition should be changed to the hospitalization period and the examination should be performed again, which may take a lot of time and effort.

In addition, since an input operation of the displaying request and displaying of the searching result can be performed while displaying the clinical flow screen 81, it is possible to save time and effort such as switching the screens related to the medical examination process from the searching screen and searching for a screen which is wanted to be viewed as in the method in the related art. Furthermore, since the medical examination data is shown as pictograms and the important medical examination data is displayed in the different display mode, the medical examination result and the like become easier to be recognized by the doctor and the medical examination process becomes easier to be compared with each other in a short time.

### Second Embodiment

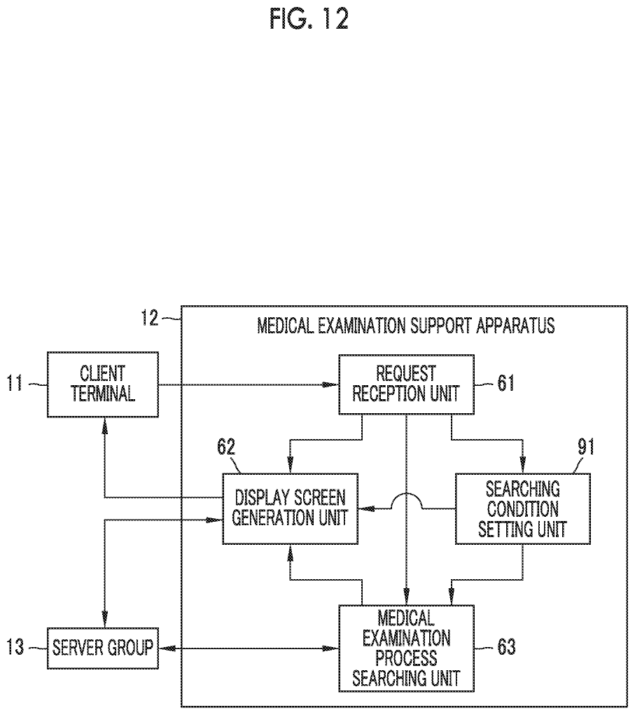

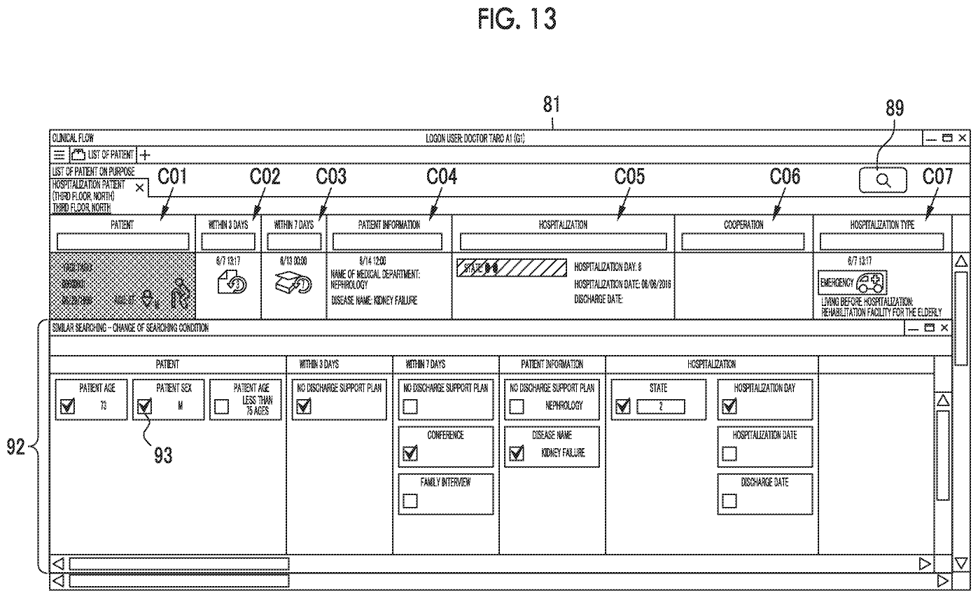

In the first embodiment described above, the searching process is performed by using the preset searching condition. However, as shown in FIG. 12, a medical examination support apparatus may comprise a searching condition setting unit 91 which sets a searching condition, and the searching condition set by the searching condition setting unit 91 may be displayed on the display screen such that a user may input the searching condition. In this case, the display screen generation unit 62 overlaps the searching condition set by the searching condition setting unit 91 on the display screen. For example, as shown in FIG. 13, a searching condition 92 is displayed in an overlapping manner on the clinical flow screen 81.

After selecting a patient in the same manner as in the first embodiment, the searching condition 92 is displayed with the overlapping manner on the clinical flow screen 81 in a case where the user performs an input operation different from that at the time of selecting the patient, such as a right-clicking operation using the pointing device. The user can edit the searching condition 92 displayed on the clinical flow screen 81 and set the searching condition for the searching process by medical examination process searching unit 63.

In an example shown in FIG. 13, each item of the searching conditions 92 is already input in the initial state, and the initial state is input all the medical examination data of “Yagi Taro” who is the selected patient, that is, the patient-information column C01, the three-day medical examination result column C02, the seven-day medical examination result column C03, the patent-information column C04, the hospitalization-state column C05, the cooperation information column C06, the hospitalization type column C07, and the like. The user can turn on or off a check box 93 by a clicking operation using the pointing device of the client terminal 11, or can edit the searching condition 92 by inputting the information using a keyboard and the like. Then, in a case where the user wants to perform the searching process by using entire medical examination data of “Yagi Taro”, the user ends the input operation as the initial state, and in a case where the user wants to perform the searching process by using a part of the medical examination data of “Yagi Taro”, the user edits the searching condition 92 by turning off the check box 93 and the like.

As described above, after inputting the searching condition by the client terminal 11, for example, the input operation of the searching process is completed by clicking the searching button 89. In a case where the input operation of the searching process is performed, the client terminal 11 issues the searching result displaying request. The searching condition is included in the searching result displaying request and issued from the request issuing unit 42. In a case where there is a plurality of items of the medical examination data which is set as the searching condition, a value of weighting may be set for each item.

As the item which can be set by the value of weighting, for example, in a predetermined examination, the examination result in a case of sharply changing examination value can be used as the examination condition, and the value of weighting can be set. Accordingly, it is possible to search for a similar patient with similar examination result in the past, and use the result for the future medical examination process to be performed. In addition, the apparatus comprises a function of detecting signs that shows a likelihood of sharp change in the examination value in the future even though it is not judged that sharp change therein occurs. In a case of detecting such a sign, for example, the apparatus may automatically issue warning including a content of “There is a sign that an examination value XX is likely to change shapely, and thus it is recommended to set a value of weighting for corresponding searching condition YY and search for similar patients.” by voice or display on the screen. With the above warning, it is possible to predict a sharp change in a patient and take measures in advance. In this case, for example, from the example of the symptom where the examination value has shapely changed in the past, conditions which are regarded as the sign can be obtained by a method of machine learning and an examination result that matches with the condition can be detected.

The request reception unit 61 inputs the searching condition issued together with the searching result displaying request from the client terminal 11 to the searching condition setting unit 91 in a case of receiving the searching result displaying request from the client terminal 11. Accordingly, a searching condition at the time when the searching is performed by medical examination process searching unit 63 is set. Then, the searching condition is updated by receiving each searching result displaying request from the client terminal 11. The medical examination process searching unit 63 performs the searching process by using the searching condition set by the searching condition setting unit 91. Then, the searching result can be acquired in the same manner as in the first embodiment and can be displayed on the display screen such as the clinical flow screen 81.

As described above, since the searching condition 92 can be overlapped on the clinical flow screen 81 and the user can edit the data in the same screen, it is possible to save time and effort such as switching screens and looking for a searching screen. In addition, in a case where the value of weighting is set for each item of the medical examination process and there are a plurality of medical examination processes with the same overall similarity, a medical examination process including medical examination data having a high similarity in items set as a high value of weighting may be displayed in the upper rank of the searching results.

### Third Embodiment

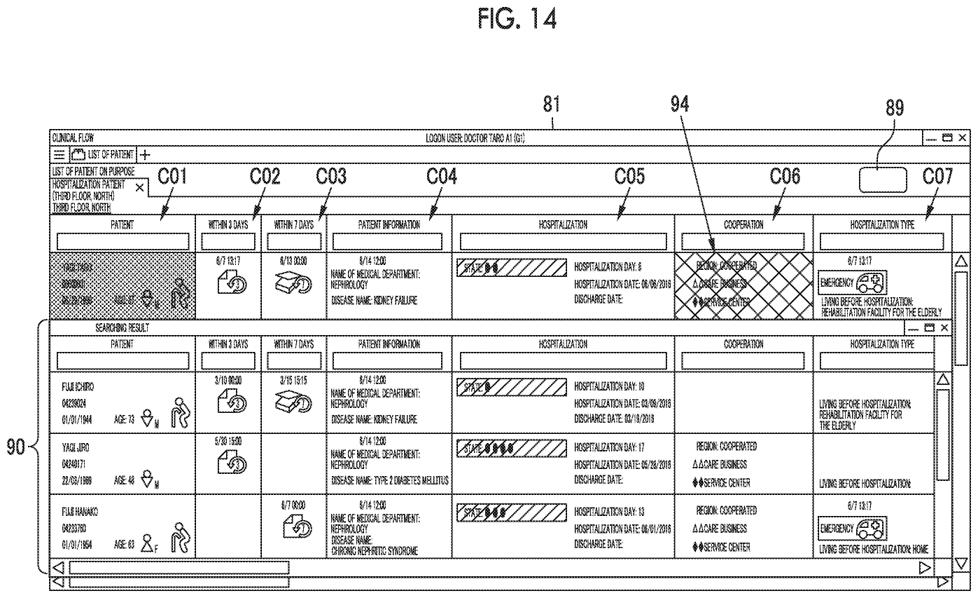

In the each embodiment described above, a searching result is displayed in an overlapping manner on the display screen after searching for medical examination process of similar patients. However, a process performed after the searching is not limited to the displaying of the searching result, and, as shown in FIG. 14, in a medical examination process of a selected patient, a prediction of process of a future medical examination to be performed may be made and a prediction result 94 may be displayed on a display screen. As a prediction method, the searching process is performed in the same manner as in the each embodiment described above, and medical examination data included in the medical examination process of a similar patient having high similarity with the medical examination of the selected patient is used as the prediction result 94. In the present embodiment, the state before performing the searching process is the same state in the clinical flow screen 81 shown in FIG. 8. In the example shown in FIG. 8, a display field of the cooperation-information column C06 is blank in a medical examination process of “Yagi Taro” who is the selected patient.

Then, in a case where the searching process is performed in the same manner as in the each embodiment described above, the prediction result 94 is input to the display field of the cooperation-information column C06 which is blank together with the searching result 90. In an example shown in FIG. 14, since a medical examination process of “Fuji Ichiro” having the highest similarity to a medical examination process of “Yagi Taro” who is the selected patient has blank in the display field of the cooperation information column C06, medical examination data of a cooperation information column C06 included in a medical examination process of “Yagi Jiro” having the next highest similarity is used as the prediction result 94.

In addition, in the example shown in FIG. 14, in order to recognize the prediction result 94, the display field of the cooperation information column C06 is displayed in a different display mode. In FIG. 14, although a difference in the display mode is represented by presence or absence of a shaded pattern for convenience of drawing, the display mode is not limited thereto. The display mode may be a display mode which can allow the display field of prediction result to be distinguished from other display fields.

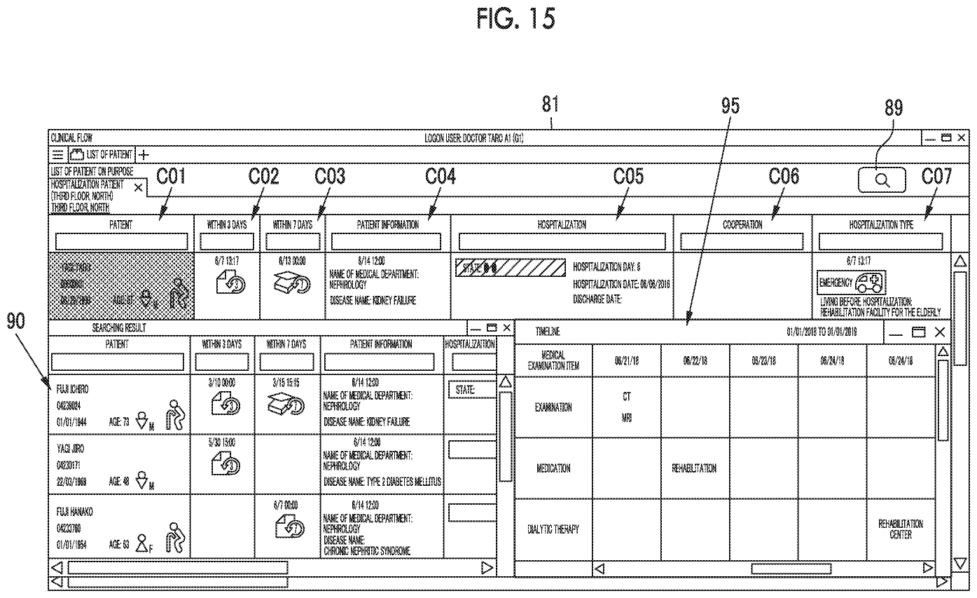

In addition, the prediction result of the medical examination process not only may fill the blank of the display field as shown in FIG. 14, but also may display a timeline 95 as the prediction result as the example shown in FIG. 15. The timeline 95 is displayed in an overlapping manner on the display screen after performing the same searching process as that in the each of embodiments described above. The timeline 95 is a timeline in which a part or entirety of the medical examination process of the selected patient is displayed in time series. In an example shown in FIG. 15, the timeline 95 as a prediction result is displayed to be overlapped on the clinical flow screen 81 together with the searching result 90. Similarly to the example of the prediction result 94 as shown in FIG. 14, the timeline 95 is created based on the medical examination data included in the medical examination process of the similar patient having a high similarity with the medical examination of the selected patient.

In addition, the prediction result of the medical examination process is not limited to the example shown in FIGS. 14 and 15, and may predict a discharge destination of the selected patient. As the prediction method of the discharge destination, for example, in a case where there is data of discharge destination in the medical examination data included in the medical examination process of the similar patient having a high similarity with the medical examination of the selected patient, the data is used as the prediction result.

In each of the embodiments described above, the hardware structures of processing units for executing various kinds of processing, such as the GUI controller 41, the request issuing unit 42, the request reception unit 61, the display screen generation unit 62, the medical examination process searching unit 63, and the searching condition setting unit 91, are various processors, which are exemplified below. Examples of various processors include a central processing unit (CPU) that is a general-purpose processor that executes software (program) to function as various processing units, a graphical processing unit (GPU), a programmable logic device (PLD) that is a processor where its circuit configuration can be changed after manufacturing, such as a field programmable gate array (FPGA), and a dedicated electric circuit that is a processor having a circuit configuration that is specially designed in order to execute various kinds of processing.

One processing unit may be implemented by one of various processors, or may be implemented by combining two or more processors of the same type or different types (for example, a combination of a plurality of FPGAs, a combination of a CPU and an FPGA, or a combination of a CPU and a GPU). Alternatively, a plurality of processing units may be implemented by one processor. As an example of implementing a plurality of processing units using one processor, first, as represented by a computer, such as a client computer or a server computer, there is a form in which one processor is implemented by a combination of one or more CPUs and software and this processor functions as a plurality of processing units. Second, as represented by a system on chip (SoC) or the like, there is a form of using a processor that realizes the function of the entire system including a plurality of processing units with one integrated circuit (IC) chip. Thus, various processing units are configured by using one or more of the above-described various processors as a hardware structure.

More specifically, the hardware structure of these various processors is an electrical circuit (circuitry) in the form of a combination of circuit elements, such as semiconductor elements.

### EXPLANATION OF REFERENCES

10: medical examination support system
11: client terminal
12: medical examination support apparatus
13: server group
14: network
21: electronic medical record server
21A: medical record database
22: image server
22A: image database
23: report server
23A: report database
32: memory
33: storage
34: communication unit
35: connection circuit
36: display unit
37: operation unit
39: operating program
41: GUI controller
42: request issuing unit
52: memory
53: storage
54: communication unit
55: connection circuit
59: operating program
61: request reception unit
62: display screen generation unit
63: medical examination process searching unit
66: display controller
71: initial screen
72: schedule display field
73: e-mail display field
74: list display field
78: scroll bar
79: scroll bar
81: clinical flow screen
81: display screen
82: clinical flow display field
83, 84, 85, 86, 87, 88: pictogram
89: searching button
90: searching result
91: searching condition setting unit
93: check box
94: prediction result
95: timeline

What is claimed is:

1. A medical examination support apparatus comprising:
a display screen generation unit that generates a display screen for displaying identification information of a patient and a medical examination process so as to be associated with each other for each of a plurality of patients; and
a medical examination process searching unit that compares a medical examination process corresponding to a selected patient selected from the plurality of patients to a medical examination process corresponding to a patient other than the selected patient and searches for a medical examination process corresponding to a similar patient, which has a high similarity to the medical examination process corresponding to the selected patient,
wherein the display screen generation unit displays a searching result by the medical examination process searching unit on the display screen.

2. The medical examination support apparatus according to claim 1, further comprising:
a searching condition setting unit that sets a searching condition in a case of searching by the medical examination process searching unit and inputs the searching condition to the medical examination process searching unit,
wherein the display screen generation unit displays the searching condition set by the searching condition setting unit on the display screen.

3. The medical examination support apparatus according to claim 2,
wherein the display screen generation unit displays medical examination data configuring the medical examination process on the display screen,
the searching condition setting unit sets the medical examination data as the searching condition, and
the medical examination process searching unit searches for the medical examination process including medical examination data having a high similarity to the medical examination data set as the searching condition.

4. The medical examination support apparatus according to claim 3,
wherein the medical examination process searching unit performs the searching by using similarity between pieces of medical examination data, the similarity being set by machine learning.

5. The medical examination support apparatus according to claim 3,
wherein the medical examination data includes a process of future medical examination scheduled to be performed and a result of past medical examination performed.

6. The medical examination support apparatus according to claim 3,
wherein the display screen generation unit generates a pictogram indicating the medical examination data and displays the pictogram as the medical examination data displayed on the display screen.

7. The medical examination support apparatus according to claim 3,
wherein the display screen generation unit makes important medical examination data displayed in a different display mode than that of other medical examination data in the medical examination process.

8. The medical examination support apparatus according to claim 1,
wherein a process of medical examination for the selected patient is predicted by using the searching result from the medical examination process searching unit.

9. The medical examination support apparatus according to claim 1,
wherein a timeline for displaying a part or entirety of the medical examination process of the selected patient in a time series is created by using the searching result from the medical examination process searching unit.

10. The medical examination support apparatus according to claim 1,
wherein a discharge destination of the selected patient is predicted by using the searching result from the medical examination process searching unit.
